(19) United States
(12) Patent Application Publication
(10) Pub. No.: US 2012/0189710 A1
(43) Pub. Date: Jul. 26, 2012

(54) ANTIBACTERIAL COMPOSITIONS

(76) Inventor: Raymond S. Steggles, Worcester (GB)

(21) Appl. No.: 13/387,434

(22) PCT Filed: Jul. 28, 2010
(86) PCT No.: PCT/GB2010/001434

§ 371 Date: Apr. 10, 2012

Related U.S. Application Data
(60) Provisional application No. 61/229,077, filed on Jul. 28, 2009.
(60) Provisional application No. 61/289,080, filed on Dec. 22, 2009.

(51) Int. Cl. A61K 31/65; A61P 1/02; A61P 31/04; A61K 33/00; A61K 31/5415

(52) U.S. Cl. 424/613; 514/154; 514/224.8

(57) ABSTRACT

Compositions and their use for treating bacterial infections are described, in particular compositions for treating multi-species infections and the treatment of bacterial infections arising from biofilms. The present invention also relates to antibacterial compositions for treating dental diseases. Compositions of the present invention may include a garlic extract or S-allyl cysteine, and optionally an antibiotic and/or an antiseptic.

### FIELD OF THE INVENTION

The present invention relates to compositions with antibacterial effect, particularly those useful for treatment of humans and for treatment of infections and conditions caused or promoted by multi-species biofilms. The present invention further relates to compositions useful for treating dental infections and diseases.

### BACKGROUND OF THE INVENTION

Bacterial colonies and infections contribute significantly to a number of health problems. Bacterial cells may form a biofilm, a collection of bacteria living on a surface such as skin, internal mucous membranes, open wounds, soil, roots of plants and so on. Antibiotics (that is, antibacterial agents) are often employed to treat bacterial infection. However, the use of systemic antibiotics has several disadvantages. There is high incidence of gastro-intestinal side effects, and an increased risk of developing antibiotic resistant bacterial strains. Furthermore, such treatment regimes generally involve multiple doses being taken, and so are somewhat reliant on patient compliance. Antiseptics are also often used to treat bacterial infection, but suffer from the disadvantage that multiple applications are often required to remove a biofilm because only sub-lethal doses may reach the protected bacteria deep in the core of the biofilm.

Until recently, it was believed that the cells within a biofilm acted as individuals, in an isolated haphazard manner. However, it has since been recognised that particular bacterial species produce molecules detectable by others of the same species. Furthermore, it has been observed that as the concentration of these molecules increases, a threshold is reached whereafter the bacteria began to act in a similar manner. This is postulated as being caused by signalling within the biofilm, which has been referred to as quorum sensing.

Research into this signalling has increased as more bacteria are identified as producing and/or responding to similar signals. It appears that most bacteria can do this.

There are two major groups of bacteria, Gram negative and Gram positive. They appear to have a common signalling system, such that response can be observed in bacteria to the presence of the primitive signal. This signal is known as Autoinducer 2. Additionally, each of the two major groups has different species and even strain specific signals. Gram negative bacteria use a group of signals known as Autoinducer 1, which are N-acyl homoserine lactones (AHLs) of various configurations. Gram positive bacteria use more specific short-chain oligopeptide molecules.

Research is ongoing into identification of specific inter-strain or inter-species signalling molecules of recognised pathogens and ways to block them.

Some investigation (Bjarnsholt, T. et al, Microbiology (2005), 151, pp. 3873 to 3880) into this area of quorum sensing has identified Pseudomonas aeruginosa as a quorum sensing bacterium, which sensing was postulated as being blocked by a garlic extract. In this study, garlic extract was administered to mice for two days as a prophylactic treatment, following which Pseudomonas aeruginosa was introduced. Treatment with garlic extract continued, in combination with tobramycin. The dosage of garlic extract given was 1.5% of the mass of the mouse, every 24 hours for seven days. However, these studies have not found compositions suitable for human use. Garlic extracts separated using toluene are unsuitable because of toluene's carcinogenic breakdown products. The dosage required is also extremely high—it has been noted that an 80 kg person would have to ingest 50 whole bulbs of garlic every day. Furthermore, the treatment period is extensive—the garlic extract was administered for two days as a prophylactic before any Pseudomonas aeruginosa bacteria were introduced, and treatment continued for seven days.

Further difficulties arise in more complex bacterial environments. For example, although studies relating to use of garlic extracts against single species biofilms are known, multi-species environments such as those found orally have not been considered a viable target. Multi-species diseases such as dental diseases such as root canal infection and periodontal disease, wound infections, bedsores and ulcers, in which multiple species of bacteria have invaded tissue, are therefore difficult to treat successfully.

Potential uses of allium related compounds (that is, compounds containing one or more organosulfur groups that can form, via metabolism or otherwise, membrane permeable small molecular thiols containing 5 or fewer carbon atoms, said thiols being oxidised in vivo to form membrane permeable disulfides or mixed sulfides, and said disulfides or mixed disulfides being capable of being further oxidised in vivo to form membrane permeable thiosulfinates or mixed thiosulfinates; this does not includes organosulfur amino acids or organosulfur peptides because these are not membrane permeable), such as anti-inflammatory, antioxidant and antiarsenicosal, have been postulated in WO 2006/127918. That document also describes use of allyl mercaptan, a metabolite of allicin, as an ingredient in an antimicrobial toothpaste.

Periodontology is a branch of dentistry studying the support structures of the teeth. Periodontal diseases contribute greatly to the incidence of tooth loss in adults, and are often the result of bacterial infection and biofilm formation. About 90% of the population will be affected by periodontal disease during their lifetime to a greater or lesser extent. Around 15% are severely affected, some at a very young age. The disease often progresses sufficiently that dentures are required—this is thought to be the case for around 20% of the population.

Dental plaque consists of tens, possibly hundreds of species, including both Gram negative and Gram positive bacteria. As noted above, Gram positive bacteria do not use AHL signalling systems. Known periodontal pathogens include Porphyromonas gingivalis, Aggregatibacter actinomycetemcomitans, Tannerella forsythensis, Prevotella intermedia, Treponema denticola, and Fusobacterium nucleatum. Dental decay is caused by very few bacterial species, mainly Streptococcus mutans and in the later stages by Lactobacilli species. Each of these bacterial species has been discovered to use AI 2 signalling pathways.

The species specificity of AHL signals and receptors within the Gram negative group presents a very complex signalling picture in multi-species, Gram negative and Gram positive bacteria containing environments.

Dental diseases such as root canal infection and periodontal disease are further complicated by the spectra of conditions interacting. Firstly, the nature and composition of the biofilm involved. Prior to disease development the biofilm may be one which is protective, but may develop into one which is highly pathogenic. Research is directed towards ways of eliminating specific bacteria believed to be the main contributors to disease. It is normal for biofilm to exist on gum surfaces, but it is abnormal for pockets to exist and be colonised by bacteria, although as we age it becomes more common for this abnormal state to occur. The presence of colonised pockets is now believed to be detrimental to general health, contributing to other conditions such as cardiovascular disease, lung infections, renal disease and diabetes among others.

The second complication is the immune response, which may be highly protective, preventing tissue damage, or may be overactive, producing self-harm by destroying tissue. This overactive response is centre of much interest. It is believed that the major factor in bone loss is the immune response to bacterial invasion. Bone loss eventually causes tooth mobility, loss of teeth and the requirement of dentures to maintain limited function and restore appearance.

A final problem is that of the varying degree of oral hygiene measures carried out. In general it is recognised that disruption of dental plaque (a biofilm) reduces the likelihood of developing advanced periodontal disease. Disruption forces the remaining bacteria to re-organise, preventing them from developing the organisational conditions to become pathogenic. Many consumer products are directed to this area, with limited long-term clinical results. Where the disease has become established, the gum separates from the tooth surface leaving pockets where the biofilm can exist and be relatively undisturbed by any oral hygiene measures.

Treatment of bacterial infection in dental applications is therefore of great importance to improve oral health. Control of periodontal disease is possible with very careful attention to oral hygiene. However, periodontal disease gives few symptoms until the very late stages, when treatment becomes difficult.

The generally used treatment method is careful mechanical cleaning by a dentist or hygienist. This is inadequate for 30% of the population, whose condition deteriorates requiring further intervention involving topical antibiotics, antiseptics, or anti-inflammatories, and/or systemic antibiotics, or anti-inflammatories. Even these treatments fail 20% of the population, despite the disease being slowed.

Application of mechanical and antibiotic techniques fails to provide long term control of periodontal and other dental diseases. One current view is that periodontal pockets are recolonised by fresh bacteria from the oral cavity before the gum can heal after treatment. It has been postulated that an unrecognised immune deficiency allows quick recolonisation and re-establishment of disease. Another, alternative, view is that failure to completely eradicate the existing biofilm permits remnants to rapidly re-establish and resume activity, even in individuals without any identifiable immune deficiency.

So, it is clear that there is a great need for improved methods for treating dental diseases, particularly periodontal disease.

### SUMMARY OF THE INVENTION

The present invention seeks to provide compositions with antibacterial action. The present application is founded on the surprising realisation that quorum sensing inhibition can be used to treat complex infections, more complex than that treated with garlic extract and tobramycin by Bjarnsholt et al. Bjarnsholt described only treatment of a single species infection, introduced after days of prophylactic treatment by an impractically high dosage of medicament. The present inventor has found that more practical therapies are possible, against a much broader range of conditions.

At its broadest, the present invention relates to compositions and methods for treating bacterial infections. Particularly, the present invention relates to compositions for treating multi-species infections, which can include Gram negative and Gram positive bacteria, or different species which are solely Gram negative or Gram positive. The present invention finds particular application in the treatment of bacterial infections arising from biofilms. The present invention also relates to antibacterial compositions for treating dental diseases. Compositions of the present invention may include a garlic extract or S-allyl cysteine, and some compositions do not need to include an antibiotic and/or an antiseptic.

Other of the compositions and methods of the present invention use a combination of a quorum sensing inhibitor with an antibiotic and/or an antiseptic.

Accordingly, in one aspect the present invention relates to a composition for treating a multi-species bacterial infection, the composition comprising: (i) a quorum sensing inhibitor; and (ii) an antibiotic and/or (iii) an antiseptic. This use is not expected from Bjarnsholt, which only suggests utility against a particular single species bacterial infection. Furthermore, Bjarnsholt does not disclose the composition or use of a quorum sensing inhibitor in combination with an antiseptic; or the composition or use of a quorum sensing inhibitor, antiseptic and an antibiotic.

In such compositions, the quorum sensing inhibitor preferably blocks the action of the N-acyl homoserine lactone (AHL) family of signalling molecules. The quorum sensing inhibitor is preferably a garlic extract. In some embodiments, the garlic extract is water soluble. Preferably, the garlic extract is at least partially fibrous.

Some preferable compositions according to the present invention comprise 2 mg of the garlic extract. The garlic extract used in such relatively small dosage is a great contrast to that suggested by Bjarnsholt, which would be equivalent to an 80 kg person ingesting 50 whole bulbs of garlic every day

The quorum sensing inhibitor used preferably comprises S-allyl cysteine. In some embodiments, the quorum sensing inhibitor is S-allyl cysteine.

Preferably, the antibiotic used has chelating activity. Preferably the antibiotic is a tetracycline antibiotic such as tetracycline or minocycline. A particularly preferred antibiotic is tetracycline. Preferable compositions of the present invention contain 250 mg of the antibiotic. In preferable embodiments, the antibiotic is in particulate form. This allows the antibiotic to chelate in the infected area, prolonging its activity there and increasing its effect.

The antiseptic may be active in the composition or be a precursor compound which requires activation to become an antiseptic. A particularly preferred active antiseptic is chlorohexidine digluconate. Preferred compositions of the present invention contain 2.5 mg in the form of a chip (PerioChip®), one of which is applied to each periodontal pocket, or as a gel formulation containing 1% w/w chlorohexidine digluconate. The preferred precursor compound is tolonium chloride used in the well established Photo-Activated Disinfection (PAD) system.

In some embodiments of the present invention, the composition further comprises glycerine, in some embodiments 0.7 ml of glycerine. The composition may comprise water, in some embodiments one or two drops of water. In preferred embodiments the composition is a paste. In other embodiments, the composition may be a toothpaste, a mouthwash, or a chewing gum.

The multi-species infection may comprise a mixture of Gram negative and Gram positive bacteria. Alternatively, it may consist of several different Gram negative bacteria, or several different Gram positive bacteria. Preferably, the infection comprises Gram negative bacteria. In preferred embodiments of the present invention, the multi-species bacterial infection is a bacterial dental infection. By dental, it is taken to mean any part of the oral cavity, not simply the teeth. So, infections of the roots, gums and so on are taken to be covered by this term. More particularly, the bacterial dental infection may be periodontal disease. Such diseases are quite different to that treated in Bjarnsholt, a single species lung infection. In an alternative embodiment, the composition may be coated or applied to the surface of medical apparatus the comes into contact with potential sites in the body where multi-species infections may reside. By way of example, the compositions disclosed herein may be applied to catheters for preventing catheter induced infection. As catheters generally have a gelatinous film applied to them to ease insertion, the compositions of the present invention can conveniently be added to such compositions. In a further possibility, the compositions of the present invention may be employed for the treatment or prevention of urinary tract infection, for example, by topical application, where a systemic approach is proving ineffective.

A second aspect of the present invention provides use of a composition as described in the first aspect in the manufacture of a medicament for treating a multi-species bacterial infection. Preferably, the multi-species bacterial infection is a bacterial dental infection.

In all aspects of the present invention, examples of bacterial dental infections that have been successfully treated by the present invention include the treatment of periodontal disease, by way of therapeutic and/or prophylactic treatment. By way of example, the periodontal disease may be (a) the treatment or prevention of gingivitis or periodontitis; (b) the treatment of pericoronitis or the inflammation around wisdom teeth; (c) the treatment of peri-implantitis or the inflammation of bone around a dental implant; (d) endodontic treatment or the treatment of inflamed pulp tissue; (e) treatment of a failed apicectomy; (f) post-extraction preventative treatment of a tooth socket to prevent the development of infection and localised osteitis; and/or (g) treatment following apicectomy, applying the composition to the bone and the underside of the gum flap prior to suturing.

A third aspect of the present invention provides a method of treating a multi-species bacterial infection, comprising the steps of: (1) applying a quorum sensing inhibitor to the multi-species bacterial infection; and (2) applying an antibiotic to the multi-species bacterial infection and/or (3) applying an antiseptic to the multi-species bacterial infection. In some embodiments, steps (2) and/or (3) are carried out either after step (1) or at the same time as step (1).

In preferable methods, steps (2) and/or (3) are carried out at the same time as step (1).

The methods of the present invention may further comprise the initial step of applying citric acid to the multi-species bacterial infection; said initial step being carried out before step (1). Preferably, the citric acid is applied for a period of approximately two minutes.

In preferable methods of the present invention, steps (1) and (2) and/or (3) are carried out at the same time by application of a composition according to the first aspect described above to the multi-species bacterial infection. In some embodiments the composition is applied for approximately 4 minutes. In the case of chlorhexidine digluconate chips, the quorum sensing inhibitor containing paste is syringed into the pocket, followed by immediate placement of the chip remaining within the pocket whilst they dissolve and act locally on remaining biofilm.

In some embodiments, the multi-species bacterial infection is a bacterial dental infection. More particularly, the bacterial dental infection may be periodontal disease.

The present invention also relates to a method of treatment of a patient in need thereof, comprising the steps of: (1) applying a quorum sensing inhibitor to the patient; and (2) applying an antibiotic to the patient and/or (3) applying an antiseptic to the patient. In some embodiments, steps (2) and/or (3) are carried out either after step (1) or at the same time as step (1). The patient is preferably an animal, more preferably a human. The patient is generally suffering from a multi-species bacterial infection, preferably a dental disease, and more preferably periodontal disease.

Embodiments of the present invention will now be described in more detail by way of example and not limitation with reference to the accompanying figures.

### BRIEF DESCRIPTION OF THE FIGURES

FIG. 1 comprises X-Ray scans (left) and bone density plots (right) taken during a confidential clinical trial in which a patient was treated with a composition according to the present invention. FIG. 1a shows the condition of the bone at Day 0 (before treatment on Day 0), FIG. 1b shows the condition of the bone at Day 42, and FIG. 1c shows the condition of the bone at Day 356.

FIG. 2 shows changes in bone height around the mesial (front, towards the other teeth) and distal (towards the back of the mouth) roots of the tooth of the patient of FIG. 1. The tooth in question has two mesial roots, superimposed on the X-Ray of FIG. 1, and one distal root. The graph shows bone height before the treatment was carried out on Day 0 and after 1 year.

FIG. 3 shows the same data as FIG. 2, in terms of the percentage of the tooth which is in the bone. Again, the data are represented for the mesial (front, towards the other teeth) and distal roots of the tooth of the patient of FIG. 1, showing data for before the treatment was carried out on Day 0 and after 1 year.

With reference to FIGS. 2 and 3, “mes-ant”, “mes-post”, “dist-ant” and “dist-post” refer to the anterior border of the mesial roots, the posterior border of the mesial roots, the anterior border of the distal root and the posterior border of the distal root respectively.

FIG. 4 shows diagrammatically the postulated steps of previously known antibiotic treatment of a biofilm, and the inadequate result.

FIG. 5 shows postulated steps in the action of the present invention against a biofilm.

### DETAILED DESCRIPTION

### 1. Quorum Sensing Inhibitor

The quorum sensing inhibitor used in the present invention interferes with inter-bacterial signalling in the biofilm to be treated. Preferably, it is a quorum sensing inhibitor which interferes with AHL signalling. In some embodiments, the quorum sensing inhibitors is an extract of garlic. Without wishing to be bound to any particular theory, it is postulated that S-allyl cysteine can act as a quorum sensing inhibitor by blocking and/or competing in the AHL signalling pathway. So, the garlic extract or quorum sensing inhibitor preferably contains S-allyl cysteine. Accordingly, the present invention provides in a further aspect a composition for treating a multi-species bacterial infection, or an infection-forming bacterial biofilm, comprising a garlic extract and an antibiotic and/or an antiseptic.

Blocking AHL signalling is thought to create a mismatch in bacterial appreciation of their environment. This disrupts the normal quorum sensing induced coordinated behaviour. Biofilm formation, growth and virulence depend on such coordinated behaviour and action, and so disruption of biofilms existing in a pathogenic condition is highly advantageous.

Exemplary members of the N-acyl homoserine lactone (AHL) family of signalling molecules have the following structures, wherein R¹ represents a chain of between 1 and 10 carbon atoms:

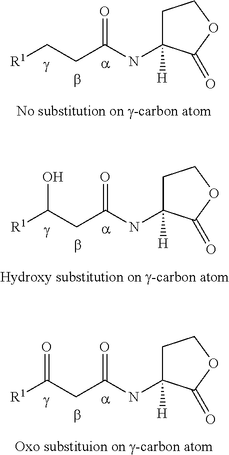

Strain specificity of AHL signalling is though to derive from variations in the group —C_{β}H₂—C_{γ}X—R¹ (that is, the acyl chain), with the rest of the molecule being common between signalling compounds.

Through molecular modelling, the compound S-allyl cysteine (SAC) has been identified which has a structural similarity to part of the AHL molecules. Without wishing to be bound by the theory, it is postulated that this structural similarity allows SAC to bind bacterial AHL receptors in place of the natural AHL molecule and/or to compete for sites with AHL precursors on enzyme producing AHL's.

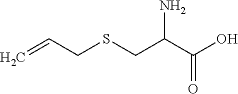

It is further postulated herein that SAC's size and similarity to AHL molecular structure allows it to pass through membranes, allowing it to interact with intra-cellular as well as surface based AHL receptors. Surface based AHL receptors tend to be those for longer chain AHLs which do not diffuse into cells, whereas intra-cellular receptors only detect diffusible short chain AHLs. As it is postulated that SAC can bind to both surface based and intra-cellular receptors, it is thought that the molecule can serve as a wide acting AHL signalling inhibitor.

Accordingly, a further aspect of the present invention provides a composition for treating a multi-species bacterial infection, or an infection forming bacterial biofilm, comprising S-allyl cysteine and an antibiotic and/or an antiseptic.

SAC is a component known to be present in garlic, and is stable for prolonged periods.

SAC is water soluble, and so garlic extracts which are water soluble are likely to contain a higher concentration of SAC than liquidised garlic or oil soluble garlic extracts. Odourless garlic extracts tend to be water soluble-oil soluble extracts of garlic being pungent and irritant.

It is postulated therefore that extracts of garlic which comprise SAC are active as quorum sensing inhibitors. Commercially available extracts have been identified which are odourless and comparatively taste free—these properties are indicative of a higher concentration of SAC, and correspondingly lower concentrations of other volatile components. It has been found that other, more odourous extracts have an irritative effect on human gums, whereas the odourless extracts have no noticeable effect.

Particular odourless garlic extracts can be obtained from various household product suppliers.

Preferably the garlic extract is at least partially fibrous. This means that it can become intertwined within chelated antibiotic particles in a target area, such as a periodontal pocket, so that the extract is retained in the pocket for an extended period of time. This allows for an extended period of quorum sensing inhibition, further enhancing the effect of the composition on the target biofilm.

The garlic extract itself may be gelatinous, or otherwise liquefied. Fibres can then act as a scaffold to adsorb or absorb the extract (for example SAC), from which the extract can diffuse or otherwise dissipate.

Preferred compositions of the present invention comprise between about 0.05% and about 10% garlic extract. More preferably, the compositions comprise between about 0.1% to about 2% garlic extract (percentages by weight of the composition).

In some embodiments of the present invention, the composition is made by mixing between about 0.5 mg and about 10 mg of the garlic extract with the other components. In preferred compositions, between about 1 mg and about 5 mg is used. Most preferably, about 2 mg is used.

### 2. Antibiotics

In general, any antibiotic suitable for human usage can be included as part of the present invention. This may include antibiotics which have previously been used systemically.

Systemic antibiotic use has several disadvantages. Firstly, there is a high incidence of gastro-intestinal side effects, as the non-targeted approach cannot distinguish between bacterial sites effectively. Secondly, there is an increased risk of antibiotic resistant strains of bacteria developing—as bacterial colonies fail to be completely destroyed, resistant cells survive and can reproduce and spread, leading to a substantially resistant bacterial strain being introduced to the population. Thirdly, systemic antibiotics are generally taken in multiple doses, spaced over an extended period of time. This means that successful treatment is reliant on patient compliance. The present invention seeks to overcome these problems.

Preferably, the compositions of the present invention are applied in such a manner that the antibiotic is held proximal to the infection for an extended period. For example, in medical applications such as treatment of multi-species infections of wounds, ulcer, bedsores etc., the composition may be incorporated into a dressing material. Particularly favoured are antibacterial agents which have chelating activity, especially in the field of treating dental diseases such as periodontal disease. This allows the antibacterial agent to be held in the target area for a longer period of time without dispersion, leading to extended ‘treatment’ time from a single application. Furthermore, chelated antibiotic may help to hold the quorum sensing inhibitor compound in the target area, again increasing the efficacy of the treatment.

Particularly suitable are antibiotics which have a broad spectrum of activity. Suitable antibiotics include the tetracycline group such as minocycline, metronidazole, penicillins such as amoxicillin and ampicillin, cephlosporins and erythromycin.

A particularly useful antibiotic is tetracycline. The antibiotic properties of tetracycline can be effective over a number of hours or even days, due to its chelating properties. Tetracycline can remain in the target area and be released slowly due to these properties.

A typical application of tetracycline is in dental care. When applied alone, it is usually ground up with water into a paste, which is soaked up into a piece of retraction cord. The cord is then introduced to the target area and left for a few minutes. The tetracycline is in particulate form generally, held in position by multiple chelation points.

This type of application of any antibiotic gives a very high local dose, with very little systemic consequences such as evolutionary pressure on gut bacteria, or other gastro-intestinal effects. By targeting antibiotic present in such a manner using, for example, chelating antibiotic, side effects caused by systemic antibiotic treatment such as gastro-intestinal effects can be reduced as the antibiotic does not travel through the host system to the same extent.

Tetracycline is a broad spectrum antibiotic, with a mechanism disrupting protein synthesis. It is bacteriostatic, being taken in actively by bacterial cells in an appropriate physiological state, although many have evolved a protective mechanism whereby they are capable of identifying it and pumping it out of the cells before metabolism is affected.

Because of the high local concentration created by this type of application, the tetracycline is capable of overwhelming the bacterial pumping mechanism. However, as with all existing antibacterial agents, it is unable to destroy the biofilm itself. The physical bulk and nature of the matrix of the biofilm can prevent spread of the active antibacterial agents. Furthermore, the bacteria generally have varying physiological states, some of which, such as the replicative state, are highly receptive to antibiotics and are easily killed, but some which, such as the dormant state, are so inactive that the cell metabolism is hardly affected by the antibiotic. These dormant type cells can survive the antibiotic treatment, and thus go on to reform bacterial colonies and the biofilm.

Preferred compositions of the present invention comprise between about 5% and about 50% antibiotic. More preferably, the compositions comprise between about 10% to about 30% antibiotic (percentages by weight of the composition).

In some embodiments of the present invention, the composition is made by mixing between about 150 mg and about 350 mg of the antibiotic with the other components. In preferred compositions, between about 200 mg and about 300 mg is used. Most preferably, about 250 mg is used. Only sufficient of the paste is applied to treat existing pockets, so the patient dose rarely reaches 250 mg, more usually being in the region of 10 mg to 50 mg.

### 3. Antiseptics

Antiseptics when used to treat bacterial infections often require multiple applications to penetrate the protected inner core of bacteria in a biofilm. The present invention seeks to overcome these problems. Prior or simultaneous application of a quorum sensing inhibitor interferes with bacterial formation and maintenance of the biofilm, diminishing the protection afforded by the biofilm and therefore allowing more effective access of the antiseptic.

In general, any antiseptic suitable for human usage can be included as part of the invention. Preferably, the compositions of the present invention are applied in such a manner that the antiseptic is held proximal to the infection for an extended period. For example, in medical applications such as treatment of multi-species infections of wounds, ulcer, bedsores etc., the composition may be incorporated into a dressing material.

Suitable antiseptics include quaternary ammonium compounds such as benzalkonium chloride, cetyl trimethylammonium bromide, cetyl pyridium chloride and benzethonium chloride; biguanidine derivatives such as chlorohexidene gluconate; oxidising agents such as hydrogen peroxide or ozone; phenolic compounds such as TCP, thymol and triclosan; iodine-containing compounds and terpene containing compounds such as tea tree oil.

The antiseptic may include a supporting compound with no antiseptic activity per se, which is activated, for example, in situ, to produce an antiseptic compound. An example of such a compound is Tolonium Chloride, which attaches to bacterial cell walls creating a point particularly vulnerable to oxidation provided by laser-induced release of oxygen from the surrounding water molecules using a system known as Photo-Activated Disinfection (PAD). The more reactive antiseptic, ozone, reacts with accessible bacterial cell walls without intermediary compounds, and may be applied directly from an ozone generation source, such as an HealOzone™ unit.

Most antiseptics kill bacteria by disrupting cell wall integrity. However, also incorporated into the present invention are antiseptics such as triclosan which interfere with bacterial metabolism and in particular the enzyme pathways required to produce AHL molecules of Gram negative bacteria.

A range of antiseptics have been incorporated into dentrifices, mouthwashes, and varnishes to control bacterial growth in the oral cavity. These are designed to prevent biofilm formation, and most provide little protection from the effects of mature biofilms. One is more effective than most, chlorhexidine digluconate, being incorporated in oral gels and mouthwashes for consumer use, and in a varnish for professional application. As adjuncts to professional cleaning chlorhexidine mouthwash may be used as an irrigant of periodontal pockets, or as a gel (1% w/w chlorhexidine digluconate, which is equivalent to 5% v/w chlorhexidine digluconate solution Ph Eur) held in position against gums for prolonged periods by loading and wearing custom made application trays. In another presentation, chlorhexidine digluconate is formed into chips containing 2.5 mg, which may be introduced into periodontal pockets.

### 4. Compositions

Preferred compositions of the present invention comprise between about 5% and about 50% antibiotic and between about 0.05% and about 10% garlic extract. More preferably, the compositions comprise between about 10% to about 30% antibiotic and between about 0.1% to about 2% garlic extract (percentages by weight of the composition).

Preferable mixtures can be made by admixing between about 0.5 mg and about 10 mg of the garlic extract, and between about 150 mg and about 350 mg of the antibiotic. More preferred mixtures include between about 1 mg and about 5 mg of the garlic extract and between about 200 mg and 300 mg of the antibiotic. Most preferably, about 2 mg of the garlic extract and about 250 mg of the antibiotic is used.

The ratio by weight of antibiotic:garlic extract in the compositions of the present invention is preferably within the range of about 700:1 to about 15:1. More preferably, the ratio is from about 300:1 to about 40:1. Most preferably, the ratio is from about 200:1 to about 100:1.

The ratio by concentration of chlorhexidine digluconate:garlic extract in the compositions of the present invention is preferably within the range 50:1 to about 5:1, more preferably 30:1 to about 10:1 and most preferably 20:1 to 15:1.

Some compositions according to the present invention further comprise a carrier. Examples of suitable carriers are water and glycerine. Some mixtures include between about 50% and about 95% glycerine. More preferably, the mixture includes between about 70% and about 90% glycerine. In mixtures including water, preferably between about 5% and about 40% water is included. More preferably, the mixture includes between about 10% and about 30% water (percentages by weight of the composition).

The amount of glycerine, water or other carrier that is included in the composition affects the properties of the resultant mixture. If a more viscous product is required, a lower proportion of carrier can be included.

If the antibiotic is included in particulate form, the size of particles may affect how much glycerine, water or other carrier must be added to achieve a given viscosity. If the particles are smaller, a lower proportion of carrier may be added to the mixture.

One particularly preferred composition is made by admixture of about 250 mg of antibiotic, about 2 mg of garlic extract and about 0.7 ml of glycerine.

Another particularly preferred composition is made by admixture of about 250 mg of antibiotic, about 2 mg of garlic extract and 1 or 2 drops of water.

Another particularly preferred composition is made by admixture of 250 mg of antibiotic, about 4 mg of garlic extract and about 0.9 ml of glycerine.

These particular compositions have a ratio of antibiotic:garlic extract of 125:1.

Compositions such as those described above are particularly suitable for treatment of multi-species infections, particularly in animals such, as humans. Compositions of the present invention are effective even after only a single application to the subject, making them much superior to previous treatments which have required regular, repeated doses. This reduction of reliance on patient compliance makes the present invention much more likely to provide successful results. Furthermore, previous treatment with garlic has involved extensive prophylactic administration before infection has occurred. The present invention can be used successfully without any need for prophylactic treatment before infection. In addition, previously very large doses have been necessary—Bjarnsholt et al describe the equivalent of 50 whole bulbs of garlic per day for an 80 kg adult. The present invention is operable at much lower dosages, making it more practical for real world treatment methods.

### 4. Postulated Mode of Action

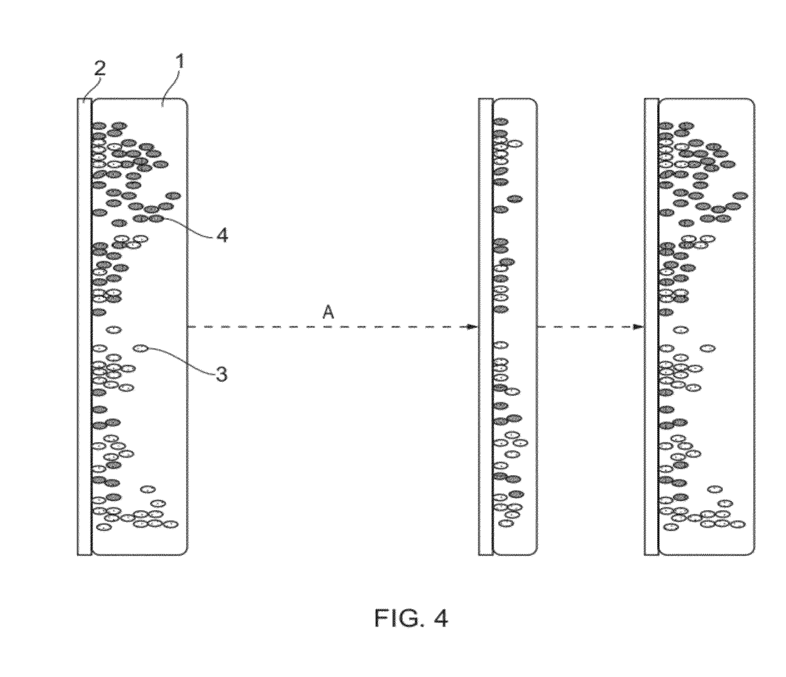

FIG. 4 shows steps of action in traditional approaches to antibacterial treatment of biofilms. A mature biofilm 1 is attached to the inflamed epithelium 2. The biofilm 1 is a sticky matrix, well organised, and so provides some protection to the bacteria against physical and chemical assaults such as antiseptics and antibiotics. The biofilm 1 comprises a large number of bacterial cells (only some of which are illustrated, for clarity). The cells are of both Gram negative 3 and Gram positive 4 groups. Within these groups there will be many different species of bacteria of differing shapes, sizes and activity. For clarity, this diversity has not been fully illustrated.

Of the cells present some will be in a dormant state (that is, having reduced activity), some will be in a replicative state (that is, undergoing cell division), and some will be in a virulence state (that is, producing virulence factors), whilst others are laying down and maintaining the biofilm matrix.

An antibacterial/antiseptic cleaning step A is performed. This step destroys a large amount of the bacterial cells 3, 4. So, in the short term, it seems that the treatment is successful. But, the treatment is not sufficient to completely destroy the bacterial colony: the protective qualities of the biofilm allow it to survive and recover. For example, dormant bacterial cells may not have been affected by the antibiotic and are free to revert to a replicative state. So, the biofilm 1 remains, albeit in a damaged form. The bacterial cells remaining in the biofilm rapidly multiply, leading to decline of the patient's condition and a return to the original infected state.

This process of decline can take three months in cases such as periodontal disease. In the case of other biofilm infections of wounds, leg ulcers and bed sores, decline can be more rapid, measured in days or even hours. In these cases less bacterial competition exists, allowing a few species to proliferate rapidly, unlike in the oral cavity where the presence of as many as 700 species delays ultimate emergence of a few.

An additional problem where some biofilm remains is caused by the patient's own immune system. The immune system is prepared to respond rapidly to bacteria present, but if the patient is genetically predisposed to overproduce harmful mediators this immuno-reaction can cause continued tissue breakdown and bone loss.

The stimuli for immune responses include molecules in the structures of bacteria, such as their cell walls, and molecules secreted by bacteria identified as foreign to the host. Under normal circumstances, once a threshold has been reached a proportionate dose response occurs, and as the stimulus declines the response reduces proportionately. If the mechanism for feedback to the immune system is disrupted, for example by host genetic variation, underlying systemic disease, or prompted by bacterial derived mediators expressed as virulence factors, the response may be disproportionately maintained for an extended period.

Host cells may produce oxidising agents which may kill invading bacteria at the expense of damaging the host's own tissue, including bone and supporting structures and fibres.

Successful destruction of the bacterial invaders permits healing to proceed, whilst failure results in continuing tissue damage. The protective nature of the biofilm allows bacteria to continue to provide stimuli for immune response, whilst remaining comparatively unaffected by that response. Meanwhile the host destroys its own tissue, unable to reach a state where healing can proceed.

It is a further advantage of using garlic or a garlic extract in the present invention that garlic has known anti-oxidative properties. These properties may aid the initial healing process by reducing host self-harm (by its immune response) whilst the immune system itself settles to normal levels.

Failure to destroy a biofilm leaves open the risk that surviving bacteria are more resistant to the antibiotic or antiseptic, and this risk increases with repeat exposure. This effect may be of particular importance if resistant strains are passed on to new hosts, allowing spread through the population. The use of systemic antibiotics can lead to such development of antibiotic resistant strains. Use of mechanical cleaning has therefore been encouraged, but complete elimination of biofilms is difficult as missing only 1 mm³ can leave 30,000,000 bacteria present. Mechanical cleaning, even at 3 monthly intervals, often means only slowed decline rather than healing.

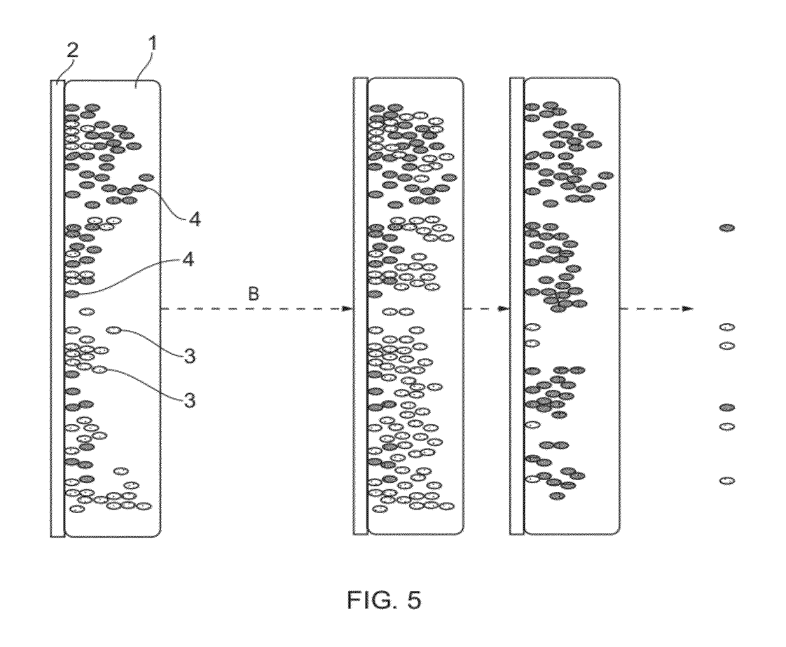

FIG. 5 shows postulated steps of action of the present invention. It is noted that this possible theory of action should not be considered binding, or in any way essential to the present invention.

As in FIG. 4, a mature biofilm 1 is present, containing bacteria 3, 4, and attached to the inflamed epithelium 2. The Gram negative and Gram positive bacteria share a common signalling (AI 2), in that both produce and detect furanosyl borate diester. The Gram positive bacteria additionally signal using AIP and membrane bound AIP receptors. Similarly, the Gram negative bacteria signal using N-acyl homoserine lactone signals and receptors, either cell wall or in the cytoplasm.

As before, of the cells present some will be in a dormant state (that is, having reduced activity), some will be in a replicative state (that is, undergoing cell division), and some will be in a virulence state (that is, producing virulence factors), whilst others are laying down and maintaining the biofilm matrix.

In FIG. 5, an antibacterial/antiseptic step B is performed, utilising not only an antibiotic but also a quorum sensing inhibitor which blocks the AHL pathway between Gram negative bacteria.

It is thought that a wide-acting AHL receptor blocker, such as a garlic extract, or S-allyl cysteine, affects the behaviour of most (possibly all) Gram-negative bacteria. In a mixed Gram-negative and Gram-positive biofilm, disruption of the Gram-negatives will substantially affect the overall nature of the mixed biofilm.

It is postulated that step B could be carried out using only a quorum sensing inhibitor, garlic extract, or S-allyl cysteine. For example some compositions may not need to include an antibiotic. Accordingly, the present invention provides a composition for treating multi-species bacterial infections, or an infection forming bacterial biofilm, comprising a quorum sensing inhibitor, a garlic extract or S-allyl cysteine. In some such compositions, the composition does not comprise an antibiotic.

With the sensing pathway between cells blocked, the Gram negative bacteria revert to behaviour that they would follow if they were individual, separated cells—that is, in their planktonic state. Once re-attached to surfaces, this behaviour changes to one of rapid cell division, to populate an area with cells. So, cells in dormant and virulence states an aspect of this being formation and maintenance of biofilm matrix) revert to replicative behaviour. This acts to increase the population of Gram negative bacteria.

However, in this replicative state the cells are also more vulnerable to antibiotic action, since they are active in uptake of compounds including the antibiotic. So, the antibiotic administered in combination with the quorum sensing inhibitor can act to destroy a very large percentage of the Gram negative bacteria.

In the case of antiseptics (not illustrated in the diagram), although uptake of antiseptics into the cell wall is passive, disruption of the matrix, resulting from the effect of the quorum sensing inhibitor, allows the antiseptic to penetrate the biofilm more thoroughly, thereby destroying more bacterial cells, whether Gram negative or Gram positive.

In the case of antibiotics mixtures this leaves a much reduced remainder population of Gram positive bacteria. These bacteria are thought then to enter their own replicative state, in an attempt to repopulate the biofilm area. Again, this makes them more vulnerable to the antibiotic, by whose action a large proportion of the Gram positive bacteria can be destroyed.

This action destroys the integrity of the biofilm, leaving isolated bacterial cells attached to the epithelium. With the biofilm sufficiently removed, the healing process can take precedence.

This approach attacks the organisation, communication, coordination and life cycles of the bacterial cells in the biofilm. By disrupting procedures common to biofilm based disease, rather than attacking a specific bacterial species as previously, the present treatment can be effective against a wide range of multi-species infections. It is thought that the present compositions and methods can be used against any biofilm based disease or infection.

At the early stage of biofilm development a small dose of AHL blocker will suffice to prevent the development of pathogenic biofilms. Garlic may have evolved to protect itself by producing substances at adequate levels to prevent pathogenic levels of bacterial colonisation.

However, if a garlic extract is applied at the same levels to a well-established disease-causing biofilm, it may not be effective alone. For example, in the present invention it may be used for the treatment of periodontal disease in combination with tetracycline. In such embodiments, the mass of the biofilm may be preliminarily reduced by mechanical cleaning. This helps to put the remaining bacteria at sufficient stress, in combination with the presence of antibiotic, that a previously ineffective dose of garlic extract becomes adequate to progress the chain of actions leading to breakdown of the biofilm and healing.

Research has shown that detection of quorum sensing is required for a range of bacterial physiological changes including their ability to form and maintain biofilms, to sequester iron (used to protect bacteria from oxygen produced by immune cells) from the host, to modify their ability to withstand acid environments, and to produce virulence factors. In the absence of quorum sensing signals, bacteria have evolved to produce large groups as soon as they can by cell division. As the bacterial group grows, quorum sensing signals increase and trigger the formation of biofilm matrix proteins and the slowing of cell division. Where bacterial growth is taking place, but quorum sensing is blocked, matrix production ceases or is defective, and/or a state of virulence is not reached.

Once in the appropriate physiological state bacteria carry on with their pathogenic activities, until they detect changes in the balance of signalling molecules in their environment, including AHLs. Sudden disruption of AHL signals by blocking with an agent such as S-allyl cysteine is likely to cause the bacteria to revert to a non-confrontational state. Presumably evolution has favoured confrontation only when the chances of success are high, and, when the converse is true, favoured remaining in a state where host tissues are unlikely to detect or respond to them.

It is therefore postulated that a sufficient dosage of a quorum sensing inhibitor, such as S-allyl cysteine, can be effective in preventing growth of a biofilm and thereby increasing the efficacy of the host's immune system against the infection, without an antibiotic necessarily being used.

It is further postulated that the strength, concentration etc. of the AHL blocker (such as a garlic extract or S-allyl cysteine) used in a composition follows an inverse relationship with the amount of other components such as antibiotics required for the composition to be effective in treatment of a biofilm or multi-species bacterial infection. At one extreme, a small amount of garlic extract, S-allyl cysteine etc. can be used in combination with a high dosage of antibiotic to form an effective composition. At the other extreme, a high concentration of garlic extract or S-allyl cysteine can be used without an antibiotic being necessary. As the effectiveness of the AHL blocker increases, the requirement for supporting actions diminishes, until control of the biofilm occurs without the requirement for antibiotics.

By attacking the integrity of the biofilm, a much greater destruction of bacteria can be achieved by the present invention than could be by previous use of, for example, systemic antibiotic treatment. This in turn greatly reduces the chances of antibiotic resistant strains developing in response to the treatment.

Persistence of the quorum sensing inhibitor and/or the antibiotic is useful for the most effective action against the biofilm. For the dormant bacterial cells to become active may take several bacterial generations. Even if the cells divide every 30 minutes, the quorum sensing inhibitor and antibiotic must be present for a period of hours for maximum effect. Preferably, the composition of the present invention includes an antibiotic which can form a chelated structure, increasing longevity in the target area, along with a quorum sensing inhibitor which can be at least partially held in the chelated structure to increase its longevity. Use of a tetracycline antibiotic and a fibrous garlic extract is one example of such a combination.

A suitable tetracycline and garlic extract paste can be present and effective in a periodontal pocket for a period of 24 hours. With bacteria dividing, for example, every 30 minutes, this means the paste can affect around 48 generations of bacteria. The chances of resistant strains being produced is low, due to the apparent biofilm destruction taking place.

Similarly, if no antibiotic is used there is no evolutionary pressure on the bacteria to evolve into a resistant strain. This approach therefore can help reduce incidence of antibiotic resistant bacterial strains, maintaining the applicability of treatment approaches which do necessitate antibiotic usage.

A similar reaction scheme can be envisaged for the use of quorum sensing inhibitors with an antiseptic, or quorum sensing inhibitor with an antiseptic and an antibiotic where exposure to a quorum sensing inhibitor causes the bacteria to revert to their replicative state, under which conditions the biofilm integrity and production fails to be maintained, in which they increase their uptake of compounds including the antiseptic, or because of biofilm breakdown they become more easily accessible. The antiseptic then targets the integrity of the cell wall of the bacteria or in the case of triclosan, interferes with cell metabolism.

### Methods

To test the compositions of the present invention, their efficacy against dental diseases, particularly periodontal disease, was investigated. It is thought that similar treatment can be achieved for a variety of other biofilm based diseases.

Before any application of the composition to a periodontal disease, mechanical root debridement was carried out using ultrasonic techniques or hand instruments. This allowed improved access for the application of the composition, for example in the form of a paste.

### 1. Application Using Retraction Cord

A 250 mg tablet of the antibiotic tetracycline was ground and mixed with the contents of a 2 mg capsule of water-soluble, odourless garlic extract. One or two drops of water are added to form the mixture into a thick paste. The paste was then soaked up into a piece of retraction cord (generally used to retract the gum from the edges of a tooth to allow a good impression to be taken for crown preparation).

The area of the mouth to which the composition was to be applied was isolated using cotton wool rolls or Drytips™, tongue guards (Linguafix™). Citric acid was applied to the exposed root surface for approximately two minutes. The patient then washed out their mouth, and the area in question was isolated again. The impregnated retraction cord was then introduced into a periodontal pocket of the patient. The cord was left for approximately 4 minutes before removal, and the patient was asked to dribble out (not to wash or spit forcefully, to minimise paste dispersal).

### 2. Application Using Syringe

A 250 mg tablet of the antibiotic tetracycline was ground and mixed with the contents of a 2 mg capsule of water-soluble, odourless garlic extract. 0.7 ml of glycerine was added and mixed thoroughly. The resultant paste was then introduced into a syringe having a blunt metal needle attached.

The area of the mouth to which the composition was to be applied was isolated using cotton wool rolls or Drytips™, tongue guards (Linguafix™). Citric acid was applied to the exposed root surface for approximately two minutes. The patient then washed out their mouth, and the area in question was isolated again. The paste mixture was then syringed into a periodontal pocket of the patient under gentle pressure until the pocket was substantially filled. The mixture was left for approximately 4 minutes before the patient was asked to dribble out (not to wash or spit forcefully, to minimise paste dispersal).

After each of these application methods, the patient was advised not to eat, drink or wash out their mouth for a further 30 minutes. Furthermore, they were instructed not to brush or floss their teeth until the following day, when normal oral hygiene measures were instructed to recommence.

Each of these application methods can be repeated as necessary in one treatment session to give the desired extent of application to periodontal pockets.

In each method, the root of the tooth may be preliminarily treated with citric acid. This treatment is generally for two minutes. The treatment with citric acid exposes calcium ions on the root surface and within the fibrils attached to the gum and tooth. This gives an enhanced ability to chelate with the tetracycline, helping retain it for an extended time. The tetracycline is generally in particulate form so that particles remain, held in place by multiple chelation points.

Preferably the garlic extract is in fibrous form so that it becomes entwined within the chelated tetracycline particles and is thus retained in the periodontal pocket for an extended period.

Immediately released oxidising agents such as oxygen released by the action of a PAD laser or ozone from an ozone generator rely on immediate penetration of the biofilm for their success. A disrupted and weakened biofilm is less resistant than a well-organised and fully structured one to the passage of the active molecules to the bacterial cell walls. Prior application of quorum sensing inhibitor, allowed to act over a period of several hours prior to introduction of such oxidising agents is beneficial, improving efficacy.

The procedure for use of the PAD laser consists of professional scaling of the periodontal pockets and application of the quorum sensing inhibitor. Because changes in biofilm organisation relies on physiological changes in the bacterial community sufficient time must be allowed for this to occur. The process of physiological change occurs within minutes, but the effects are likely to be significant only after several hours, as gradual degradation of biofilm matrix fails to be repaired. So within 24 to 36 hours tolonium chloride is introduced into the periodontal pockets and allowed to soak into the remains of the biofilm for 60 seconds. The light tip of the PAD laser is introduced into the pocket and energy applied for 60 to 90 seconds, the energy releasing oxygen from the available water molecules. In the case of ozone, again prior application of quorum sensing inhibitor 24 to 36 hours is followed by application of ozone to the pocket for 40 to 60 seconds,

Other disinfectants may be incorporated into slow release combinations with quorum sensing inhibitor containing substances, or those containing both a quorum sensing inhibitor and antibiotic combination, and applied directly to the periodontal pockets.

### Results

Over the trial period, 90 patients were treated, of whom 83 provided results for review. All of the patients had very advanced periodontal disease, and had been unsuccessfully treated with conventional treatments. Each of the patients had a single application of the composition only. The results are summarised by patient group in the following Table 1, in which: Excellent Response indicates full mouth healing with stability; Good Response indicates full mouth healing with stability in most parts of the mouth; Some signs of improvement indicates soft tissue healing, with tooth stabilisation still not under control.

| | All | Smokers | Diabetics | Males | Females |
|---|---|---|---|---|---|
| | (83) | (26) | (4) | (39) | (44) |
| Excellent | 45 (54%) | 11 (42%) | 3 (75%) | 19 (49%) | 23 (52%) |
| Response | | | | | |
| Good | 26 (31%) | 10 (38%) | 0 | 16 (41%) | 12 (27%) |
| Response | | | | | |
| Some signs | 12 (15%) | 5 (20%) | 1 (25%) | 4 (10%) | 9 (21%) |
| of improve- | | | | | |
| ment | | | | | |
| No response | 0 | 0 | 0 | 0 | 0 |
| Adverse | 0 | 0 | 0 | 0 | 0 |
| response | | | | | |

Typical effects include reduction of gum swelling and bleeding within a few days of treatment, with new bone deposition observed over several months. Healing continues going forward from treatment, often to the point of return to normal function. Teeth have become firm and stable, with reduced or clinically insignificant pocketing around the tooth. In all cases, the inflammatory response has been eliminated or substantially reduced. Whole mouth treatment of patients with widespread tooth mobility has been able to return those patients to normal eating habits.

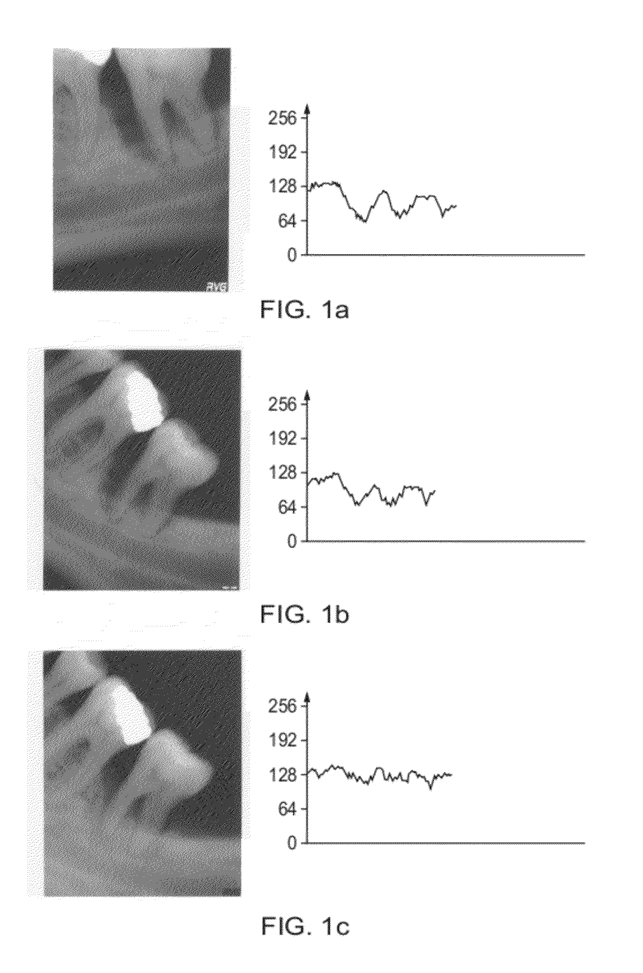

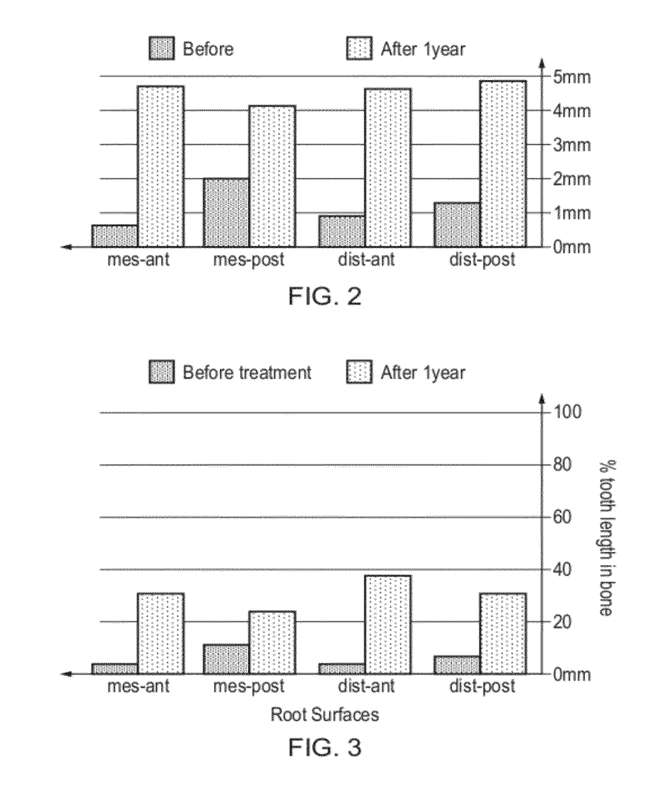

Particular data for one patient is shown are shown in FIG. 1, FIG. 2 and FIG. 3. FIG. 1 shows X-Rays (left) of the damaged area, along with bone density scans (right). As can be seen from FIG. 1a, Day 0, substantial (9 mm to 12 mm) pockets and associated bone loss had developed around the mesial and distal roots. FIG. 1b, Day 42, shows some progress in bone deposition in the affected area. FIG. 1c, Day 356, shows continued deposition, to the extent that the roots are held within deposited bone structure. The bone density scans (right) show the increase in bone density through these three stages.

FIG. 2 shows the substantial increase in bone height around the roots of the tooth. After 1 year, most of the bone heights had increased fourfold, the mes-ant height increasing nearly tenfold. The same data are presented in FIG. 3 as a percentage of the tooth length in bone. The great increase across all of the root surfaces gives rise to much improved tooth stability and function for the patient.

It is understood that the examples and embodiments described herein are for illustrative purposes only and that various modifications or changes in light thereof will be suggested to persons skilled in the art and are to be included within the spirit and scope of this application. All publications, patents, and patent applications cited herein are hereby incorporated by reference for all purposes.

1. A method for the treatment of a multi-species bacterial infection, the method comprising administering to a subject in need thereof a composition comprising (i) a quorum sensing inhibitor and (ii) an antibiotic and/or an antiseptic.

2. (canceled)

3. The method according to claim 1, wherein the multi-species bacterial infection is in the form of a biofilm.

4. The method according to claim 1, wherein the multi-species bacterial infection is a dental infection.

5. The method according to claim 4, wherein the dental infection is periodontal disease.

6. The method according to claim 5, wherein the multi-species bacterial infection is a periodontal disease is selected from (a) the treatment or prevention of gingivitis or periodontitis; (b) the treatment of pericoronitis or the inflammation around wisdom teeth; (c) the treatment of peri-implantitis or the inflammation of bone around a dental implant; (d) endodontic treatment or the treatment of inflamed pulp tissue; (e) treatment of a failed apicectomy; (f) post-extraction preventative treatment of a tooth socket to prevent the development of infection and localised osteitis; and/or (g) treatment following apicectomy, applying the composition to the bone and the underside of the gum flap prior to suturing.

7. The method according to claim 1, wherein the composition is for applying to a catheter for preventing catheter induced infection or the composition is for treating of urinary tract infection.

8. (canceled)

9. The method according to claim 1, wherein the quorum sensing inhibitor blocks the action of the N-acyl homoserine lactone (AHL) family of signalling molecules.

10. The method according to claim 1, wherein the quorum sensing inhibitor is a garlic extract.

11. The method according to claim 10, wherein the garlic extract is water soluble and odourless, or wherein the garlic extract is at least partially fibrous.

12. (canceled)

13. (canceled)

14. The method according to claim 1, wherein the antibiotic has chelating activity, or wherein the antibiotic is a tetracycline antibiotic, or wherein the antibiotic is in particulate form.

15. (canceled)

16. (canceled)

17. The method according to claim 1, further comprising glycerine, or further comprising water.

18. (canceled)

19. The method according to claim 1, wherein the antiseptic is a quaternary ammonium compound, a biguanidine derivative, a phenolic compound or an oxidising agent or wherein the antiseptic contains iodine or terpene, or wherein the antiseptic is a precursor compound which requires activation to become an antiseptic.

20. (canceled)

21. (canceled)

22. The method according to claim 1, wherein the multi-species bacterial infection causes tissue damage in the host, particularly wherein the tissue damage is to the bone of the host.

23. (canceled)

24. The method according to claim 1, wherein the multi-species infection includes at least one species of bacteria selected from the group consisting of Porphyromonas gingivalis, Aggregatibacter actinomycetemcomitans, Tannerella forsythensis, Prevotella intermedia, Treponema denticola, Fusobacterium nucleatum, Streptococcus mutans and Lactobacilli species.

25. (canceled)

26. (canceled)

27. (canceled)

28. A method according to claim 1, wherein the quorum sensing inhibitor comprises S-allyl cysteine.

29. A method of treating a multi-species bacterial infection of a subject, comprising the steps of: (1) applying a quorum sensing inhibitor to the multi-species bacterial infection of the subject; and (2) applying an antibiotic to the multi-species bacterial infection of the subject and/or (3) applying an antiseptic to the multi-species bacterial infection of the subject.

30. The method according to claim 29, wherein steps (2) and/or (3) are carried out either after step (1) or at the same time as step (1), particularly wherein steps (2) and/or (3) are carried out at the same time as step (1).

31. The method according to claim 29, further comprising the initial step of applying citric acid to the multi-species bacterial infection; said initial step being carried out before step (1), particularly wherein the citric acid is applied for a period of approximately two minutes.

32. The method according to claim 29, wherein the multi-species bacterial infection is a bacterial dental infection, particularly periodontal disease.
